(19) United States
(12) Patent Application Publication
(10) Pub. No.: US 2015/0010676 A1
(43) Pub. Date: Jan. 8, 2015

(54) ACRYLAMIDE-DEGRADING SELF-CLONING ASPERGILLUS ORYZAE

(71) Applicants: UCC UESHIMA COFFEE CO.,LTD., Hyogo (JP); Ozeki Corporation, Hyogo (JP)

(72) Inventors: Kenji Ozeki, Ishikawa (JP); Motoaki Sano, Ishikawa (JP); Kazuya Iwai, Osaka (JP); Taiji Fukunaga, Osaka (JP); Yusaku Narita, Osaka (JP); Hirokazu Tsuboi, Hyogo (JP); Takayuki Bogaki, Hyogo (JP)

(21) Appl. No.: 14/375,419

(22) PCT Filed: Feb. 4, 2013
(86) PCT No.: PCT/JP2013/052498

§ 371 Date: Jul. 29, 2014

(30) Foreign Application Priority Data
Feb. 3, 2012 (JP) 2012-022290

(51) Int. Cl. A23F 5/16; A23F 5/20; C12N 9/80

(52) U.S. Cl. CPC A23F 5/163; C12N 9/80; A23F 5/204; C12Y 305/01004
USPC 426/60; 435/254.3; 435/262.5; 426/531; 426/590; 426/595

(57) ABSTRACT

Provided are self-cloning Aspergillus oryzae that expresses amidase without induction culture exhibiting high amidase degradation activity, and a method for reducing acrylamide in which this self-cloning Aspergillus oryzae is used. Self-cloning Aspergillus oryzae, which has a gene which codes a polypeptide with a specific amino acid sequence indicated in SEQ ID NO:1, or has a base sequence hybridizable to a complementary sequence of the gene encoding SEQ ID No:1 under stringent conditions, has a protein with amidase activity which the gene is expressed without induction culture, the process of reducing acrylamide by contact treatment with the above described self-cloning Aspergillus oryzae and acrylamide-containing matter, and a method of producing reduced acrylamide food or beverage.

### TECHNICAL FIELD

The present invention relates to self-cloning Aspergillus oryzae, a method of reducing acrylamide from an acrylamide-containing matter using the Aspergillus oryzae, and a method for producing a reduced-acrylamide beverage and food using the Aspergillus oryzae. More specifically, the present invention relates to self-cloning Aspergillus oryzae which can constantly produce amidase that degrades acrylamide without induction culture and a use of the Aspergillus oryzae.

### BACKGROUND ART

Acrylamide is an organic compound having a structure expressed by CH₂═CHCONH₂, is a colorless and odor-free white crystal at normal temperature, and has a property of easily dissolving into water, an alcohol and acetone. Acrylamide is stable at room temperature but is intensively polymerized to form into polyacrylamide by heating or ultraviolet rays when it is molten.

As an effect to a human, intake of acrylamide has been known to cause skin disorder, language disorder, peripheral neuritis, cerebellar ataxia, and the like. When a large amount of acrylamide is taken in from the mouth, lungs, or skin, due to occupational exposure or an accident, it has been confirmed that disorders in the central nerve and the peripheral nerve are caused as symptoms of intoxication. According to a research conducted by International Agency for Research on Cancer (IARC), acrylamide is regarded as “a substance that probably has carcinogenicity to a human (group 2A)” in the classification of cancer-causing substances. In addition, in the proposition 65 of Toxic Substances Control Act in California, USA (safe beverage and hazardous material regulation), acrylamide has been described as a substance that causes cancer or reproductive toxicity in February, 2011.

In foods, acrylamide is considered to be generated by causing an aminocarbonyl reaction (Maillard reaction) between a specific amino acid such as asparagine, which is contained in a raw material, and a reducing sugar such as fructose or glucose by a heating treatment at high temperatures such as frying, baking and roasting. In addition to this generation route, there is a possibility that a food component other than asparagine and a reducing sugar is a causative substance and a possibility of generating acrylamide from a route other than the aminocarbonyl reaction. Acrylamide is included in foods, for example, foods that are obtained by frying potatoes, baked confectioneries that contain grains as a raw material, such as biscuits, and the like, and in beverages, for example, coffee, roasted green tea, and the like. A content of acrylamide in coffee is known to be high, and acrylamide contained in a cup of coffee is considered to be about 2 μg.

It has been known that, in microorganisms, there are species which produce amidase which degrades acrylamide, and a method of degrading acrylamide in foods and beverages by use of various microorganisms has been developed so far. For example, a method of degrading acrylamide by use of Aspergillus oryzae has been known (Patent Document 1). A method of culturing filamentous fungi in order to improve ability of an acrylamide degrading activity in a short time has been also known (Patent Document 2). When acrylamide in foods and beverages is degraded by use of microorganisms, safety is questioned in genetically-modified microorganisms. Therefore, a method of screening highly acrylamide-degrading fungi without using genetically-modified microorganisms has been developed (Patent Document 3).

### PRIOR ART DOCUMENTS

### Patent Documents

Patent Document 1: JP-A-2010-183867

Patent Document 2: JP-A-2011-92185

Patent Document 2: JP-A-2010-35449

### SUMMARY OF THE INVENTION

### Problems to be Solved by the Invention

Amidase is an induction enzyme in microorganisms in the natural world and since amidase is only produced in culture in the presence of a specific amount of acrylamide, in the case of degrading acrylamide in a beverage and food, amidase is required to be expressed by induction culture of microorganisms and its application is difficult from an industrial viewpoint. Therefore, an object of the present invention is to provide Aspergillus oryzae that can express amidase without conducting induction culture and has a very high acrylamide-degrading property. Another object of the present invention is to provide a method of reducing acrylamide, a method for producing a reduced-acrylamide beverage and food, and a reduced-acrylamide beverage and food.

### Means for Solving the Problems

The present inventors have repeated intensive studies in order to solve the above-described problems and, as a result, found that the above-described objects can be achieved by acrylamide-degrading self-cloning Aspergillus oryzae, a method of reducing acrylamide from an acrylamide-containing matter by using the Aspergillus oryzae, a method for producing a reduced-acrylamide beverage and food, and a reduced-acrylamide beverage and food, which will be described below, and thus completed the present invention.

That is, the self-cloning Aspergillus oryzae that is to be a subject of the present invention is characterized by comprising a sequence which is hybridizable under stringent conditions with a gene that encodes a polypeptide having an amino acid sequence set forth in SEQ ID NO: 1, or a nucleic acid molecule including a base sequence complementary to a gene that encodes the polypeptide, and a gene that encodes a protein having amidase activity introduced therein in the state of capable of being expressed without induction culture.

Also, the gene of the present invention is characterized by being operationally connected downstream to an improved enolase promoter.

The self-cloning Aspergillus oryzae according to the present invention is characterized in that a specific activity of amidase is at least 27 μmol/min/mg or more.

The self-cloning Aspergillus oryzae according to the present invention is characterized in that an expression amount of an amidase gene is at least 2000 times or more as compared to the original strain before self-cloning in a real-time PCR method.

A method of reducing acrylamide from an acrylamide-containing matter according to the present invention is characterized by comprising a step of subjecting the Aspergillus oryzae according to the present invention to a contact treatment with the acrylamide-containing matter.

In the method described above, the Aspergillus oryzae according to the present invention can be supported on a carrier selected from the group consisting of dried gourd, cellulose, gel beads, porous glass beads, porous ceramics, and unwoven fabric. Supporting the above-described self-cloning Aspergillus oryzae on a carrier is preferable from the viewpoint of preventing a microbial cell body from being fragile in culturing.

A contact treatment according to the present invention can include a step of reciprocal shaking culture at temperatures from 25° C. or higher to 45° C. or lower. The lower limit of the temperature in the case of reciprocal shaking culture is 25° C. or higher, preferably 30° C. or higher, and more preferably 32° C. or higher. The upper limit of the temperature is 45° C. or lower, preferably 40° C. or lower, and more preferably 35° C. or lower. When the temperature in the case of reciprocal shaking culture is 25° C. or higher, the temperature does not go below the optimal temperature in an enzyme reaction, and when it is 45° C. or lower, deactivation of an enzyme does not occur.

A method for producing a reduced-acrylamide beverage and food according to the present invention includes a step of subjecting the self-cloning Aspergillus oryzae to a contact treatment with an acrylamide-containing beverage and food.

By contact treatment with Aspergillus oryzae, the present invention can provide beverage and food with a residual ratio of 50% or less acrylamide compared to untreated beverage and food.

The present invention can provide a beverage and food comprising increased amounts of 1-propanol, ethyl acetate, 2-methyl-1-butanol, isobutyl alcohol, isoamyl alcohol, ethanol and 2-pentanone respectively twice or more as compared to before treatment due to a contact treatment with self-cloning Aspergillus oryzae.

The present invention can provide coffee beverage containing 4 ppb or less acrylamide.

### Effect of the Invention

According to the present invention, acrylamide-degrading self-cloning Aspergillus oryzae having high amidase activity can be provided. By using such self-cloning Aspergillus oryzae, acrylamide can be effectively and safely degraded from a beverage and food having a high content of acrylamide, such as coffee and roasted green tea. In addition, the strain of the present invention can produce amidase without conducting induction culture and a reduced-acrylamide beverage and food can be thus industrially provided by omitting a step of induction culture.

### BRIEF DESCRIPTION OF THE DRAWINGS

FIG. 1 is a view showing a construction procedure of a vector PenoA142 used for self-cloning in Production Example 1.

FIG. 2 is a view showing a construction procedure of a pSENSelf2 plasmid used for self-cloning in Production Example 2.

FIG. 3 shows a schematic view of gene fragments for chromosomal transformation in Production Examples 4 and 5.

FIGS. 4A and 4B show results of analyses by a southern blotting method in Production Example 5. FIG. 4A shows results of an analysis in the case of using a probe that recognizes a terminator region, and FIG. 4B shows results of an analysis in the case of using a probe that recognizes a pUC118 region.

FIG. 5 shows measurement results of an amidase specific activity of self-cloning Aspergillus oryzae in Test Example 1.

FIG. 6 shows measurement results of an amidase gene expression amount of self-cloning Aspergillus oryzae by a real-time PCR method in Test Example 2.

FIG. 7 shows results of a test of acrylamide reduction in self-cloning Aspergillus oryzae in acrylamide-added water in Test Example 3.

FIG. 8 shows results of a test of acrylamide reduction in self-cloning Aspergillus oryzae in acrylamide-added coffee in Test Example 4.

FIGS. 9A and 9B show results of tests of acrylamide reduction in acrylamide-free coffee in Test Example 5. FIG. 9A shows an effect of acrylamide reduction in an acrylamide-free coffee extraction solution, and FIG. 9B shows an effect of acrylamide reduction in an acrylamide-free coffee product.

FIG. 10 shows results of a test of caffeine reduction in a coffee extraction solution in Test Example 6.

FIG. 11 shows measurement results of amounts of phosphoric acid and organic acids in a coffee extraction solution in Test Example 7.

FIG. 12 shows measurement results of amounts of chlorogenic acids in a coffee extraction solution in Test Example 8.

FIG. 13 shows results of a test of sensory evaluations in a coffee extraction solution in Test Example 10.

### MODE FOR CARRYING OUT THE INVENTION

The present invention will be described in detail hereinbelow. The present invention relates to self-cloning Aspergillus oryzae which highly expresses a gene encoding amidase that is a protein degrading acrylamide. Aspergillus oryzae that is to be a subject of the present invention may be any species of Aspergillus oryzae in the genus Aspergillus as long as it has an acrylamide degrading activity, but Aspergillus oryzae having a high acrylamide degrading activity is preferable. Examples of filamentous fungi in the genus Aspergillus include, but are not limited to, Aspergillus oryzae, Aspergillus niger, Aspergillus kawachii, Aspergillus awamori, Aspergillus saitoi, Aspergillus sojae, Aspergillus tamarii, Aspergillus glaucus, Aspergillus fumigatus, Aspergillus 25 flavus, Aspergillus terrus, and Aspergillus nidulans. Preferred is Aspergillus oryzae that has been safely ingested as a beverage and food historically.

In the present invention, “self-cloning” means that DNA that is introduced into a host is only DNA of a microorganism belonging to taxonomically the same species as the microorganism. By being certified as a self-cloning microorganism by the Food Safety Commission, a self-cloning microorganism can be used as a general food microorganism, not as “a genetically modified microorganism.” In the present invention, for example, Aspergillus oryzae that highly expresses an amidase gene by genetic modification by use of DNA derived from Aspergillus oryzae is “self-cloning Aspergillus oryzae”. An amino acid sequence of an Aspergillus oryzae-derived amidase protein is set forth in SEQ ID NO. 1 in the sequence listing. In addition, a base sequence of an Aspergillus oryzae-derived amidase gene is set forth in SEQ ID NO: 2 in the sequence listing.

In the present invention, “a nucleic acid molecule” refers to a molecule that relates to preservation of genetic information of DNA and RNA and transmission of the genetic information, and includes a gene that encodes an amino acid sequence of a specific protein or a gene homogeneous to the gene described above. The “gene” is not limited to a natural object, but includes a gene that is artificially produced. The “homologous gene” means a gene that is highly homogeneous to the above-described gene in the base sequence and refers to a gene having a homology of, for example, 80% or more, preferably 90% or more, more preferably 95% or more, and particularly preferably 98% or more. In the present invention, a gene that encodes a polypeptide made of an amino acid sequence set forth in SEQ ID NO: 1 and a gene homologous to the above-described gene include not only natural objects but can be artificially produced. The term “hybridization” in the present invention is used as defined in Sambrook et al. (Molecular Cloning. A laboratory manual, Cold Spring Harbor Laboratory Press (1989), Cold Spring Harbor Laboratory Press (1989). The “stringent conditions” are defined according to a salt concentration, an organic solvent, temperature, and other conditions. That is, stringency increases by decrease in a salt concentration, increase in an organic solvent concentration, temperature increase of hybridization, or the like. Washing conditions after hybridization also give an effect on stringency. The washing conditions are also affected by a salt concentration and temperature. For example, the stringent conditions mean conditions such as still forming hybridization after hybridizing at 65° C. under a high ion concentration of 6×SSC and then washing with 1×SSC and 0.1% SDS at 55° C. for 1 hour.

In the present invention, self-cloning Aspergillus oryzae for highly expressing a gene that encodes a polypeptide made of an amino acid sequence set forth in SEQ ID NO: 1 (hereinafter also referred to as an amidase gene or a target gene) has an Aspergillus oryzae-derived promoter sequence, amidase gene and terminator sequence. Furthermore, the self-cloning Aspergillus oryzae may have a selective marker sequence that can be favorably used in transformation. The above-described promoter may be a promoter that can function in Aspergillus oryzae, and examples thereof include, but are not limited to, promoters such as an enolase promoter, an ADH1 promoter, a phosphoglycerate kinase (PGK) promoter, an α-amylase promoter, a glucoamylase promoter, a cellulase promoter, a cellobiohydrolase promoter and an acetoamidase promoter. The enolase promoter is preferably used, for its constant high transcription ability.

In the present invention, the “modified enolase promoter” is a promoter obtained by introducing in an enolase promoter of Aspergillus oryzae with 12 tandems of the region III that is a cis element commonly present in amylase promoters of Aspergillus oryzae. The modified enolase promoter is a promoter having high transcription ability with a power of 20 times as compared to before introduction (cited reference: Biosci. Biotechnol. Biochem., 69(1), 206-208, 2005).

The target gene in the present invention can be operationally connected to the downstream (3′ terminal side) of the above-described promoter sequence. The wording of “operationally” connected means that two nucleic acid sequences are connected in a correct orientation and a correct reading frame in order to be transcribed into messenger RNA. Known genetic engineering techniques can be used for insertion, connection and removal of a nucleic acid sequence for construction of a transformation vector.

The terminator sequence of the present invention is not particularly limited as long as it has a function that terminates transcription of messenger RNA in expression of the target gene.

The selective marker sequence of the present invention is not particularly limited as long as it is a selective marker sequence that is used in production of a transformant of Aspergillus oryzae. For example, an sC marker, a niaD marker, an argB marker, an adeA marker, a ptrA marker, a pyrG marker, and the like can be used, and an sC marker is preferable on the ground that stable genetic expression can be expected since the marker can be transfected into a chromosome in a homologous way.

In the present invention, a restriction enzyme recognition sequence can be used for connection or removal of respective base sequences. By use of a restriction enzyme, a nucleic acid sequence derived from Escherichia coli is removed and production of self-cloning Aspergillus oryzae is facilitated. However, it is preferable to design a transformation vector without including an extra restriction enzyme sequence.

In the present invention, an enhancer, a splicing signal, a poly A signal, a replication origin, and the like can be added to a transformation vector.

The transformant of the present invention includes at least one expression unit for expressing the target gene in an Aspergillus oryzae cell (a promoter sequence, an open reading frame of the target gene, a terminator sequence), but may include a plurality of the expression units described above. In the present invention, a transformant with “the copy number 1” indicates that one expression unit of the target gene is transformed into a host DNA, and there could be a transformant having “the copy number 2”, “the copy number 3”, “the copy number 4”, or the copy number more than 4 depending on the number of transformed expression units.

In the present invention, “genetic transformation in a homologous state” means that the target gene is inserted in a target site in a chromosome of a host. Meanwhile, “transformation in a heterologous state” means that the target gene is inserted in a site that is not objective in a chromosome of a host by performing transformation.

For a method of transformation in the present invention, known methods including, for example, a protoplast-PEG method, a calcium-PEG method and an electroporation method can be adopted. For genetic introduction by a protoplast-PEG method, methods described in the following can be adopted: Negrutiu et al. Plant Mol. Biol. (1987) 8: 363-373 and Mathur et al. “PEG-mediated protoplast transformation with naled DNA”, Methods in Molecular Biology 82: Arabidopsis Protocols.

Inserting an expression unit including the target gene in a chromosome of Aspergillus oryzae by transformation can be confirmed by known methods such as a southern blotting method using a probe and a PCR method. In the present invention, the “probe” refers to a molecule that is designed so as to hybridize specifically to a target sequence. Examples of the probe include DNA, RNA and PNA.

A transformant obtained by the above-described genetic transformation method can express the target gene, and the target protein can be thus provided by culturing the transformant. In a method of culturing a transformant, general culturing conditions with a medium that is usually used for culturing of Aspergillus oryzae can be adopted. For example, a YPD (Yeast peptone dextrose) medium (yeast extract 1%, peptone 2%, dextrose 2%, all expressed by w/v, pH 6.5), a CD (Czapek-Dox) medium (sucrose 3%, NaNO₃ 0.3%, MgSO₄.7H₂O 0.05%, KCl 0.05%, K₂HPO₄ 0.01%, FeSO₄.H₂O 0.001%, all expressed by w/v, pH 9.0), and the like can be used, but the medium is not limited thereto. The lower limit of a culturing temperature of a transformant is 25° C. or higher, preferably 30° C. or higher, and more preferably 32° C. or higher. The upper limit of a culturing temperature of a transformant is 45° C. or lower, preferably 40° C. or lower, and more preferably 35° C. or lower. The culturing temperature of a transformant of 25° C. or higher is not less than an optimal temperature in an enzyme reaction, and the culturing temperature of 45° C. or lower does not cause deactivation of an enzyme.

An obtained target protein is appropriately isolated or purified, if necessary, and then subjected to a qualitative analysis or a quantitative analysis, but is not necessarily purified. As a purification method, known methods such as ethanol precipitation, acid extraction, high performance liquid chromatography (HPLC), medium and high pressure liquid chromatography (FPLC), cation or anion exchange chromatography, size exclusion chromatography, affinity chromatography, hydrophobic chromatography and supercritical fluid chromatography can be adopted.

An enzyme activity of an obtained amidase protein in the present invention can be calculated, for example, by adding a microbial cell body of Aspergillus oryzae cultured in the above-described YPD medium to a Mcllvaine buffer solution to which acrylamide has been previously added and reacting for a predetermined time to quantitatively determine the generated acrylic acid by HPLC. As a specific example, the following measurement method is shown. Shaking culture is conducted on Aspergillus oryzae spores with 1×10⁷ spores/mL at 30° C. and 100 rpm for 3 days using a YPD medium, and microbial cell bodies are then collected and washed, and thereafter the microbial cell bodies that are frozen by liquid nitrogen are ground with a mortar. Thereto is added a 0.1 M-Mcllvaine buffer solution (pH 7.0) in an amount of 0.4 mL, which is a twice amount of the wet microbial cell bodies, to extract an enzyme in the microbial cell bodies. To 0.4 mL of the obtained extraction solution is added 0.4 mL of a 0.1 M-Mcllvaine buffer solution containing 2000 ppm acrylamide (pH 7.0), and the resultant is reacted at 30° C. for 30 minutes, and thereto is added 0.2 mL of 0.5 N—HCl to terminate the reaction. This reaction solution is filtered with a 0.45 μm-membrane filter and an amount of a generated acrylic acid is quantitatively determined by HPLC. For the acrylamide, one manufactured by Tokyo Chemical Industry Co., Ltd. is used, LC-2010AHT HPLC system manufactured by SHIMADZU CORPORATION is used as HPLC, and CAPCELL PAK C8 manufactured by Shiseido Company, Limited is used as the column. The measurement is conducted using a 0.1% (w/v) aqueous phosphoric acid solution as a mobile phase under the measurement conditions of a column temperature of 40° C., a detection wavelength of 200 nm and a solution sending speed of 1 mL/minute. A specific activity of amidase is calculated by an acrylic acid amount generated in 1 minute per 1 mg of a protein. The specific activity of amidase of the self-cloning Aspergillus oryzae of the present invention is at least 27 μmol/min/mg or more, preferably 50 μmol/min/mg or more, and more preferably 100 μmol/min/mg or more.

An expression amount of an amidase gene of self-cloning Aspergillus oryzae obtained in the present invention can be measured by a known method, and can be measured by, for example, a real-time PCR method. According to the real-time PCR method, a relative value of an expression amount of an amidase gene that is the target gene can be measured based on an expression amount of a house-keeping gene, or the like. For a real-time PCR equipment, 7500 Real-Time PCR System (manufactured by Applied Biosystems Inc.), Light Cycler 2.0 (manufactured by Roche Ltd.), or the like, can be used, but examples are not limited to these equipments (references: Watson, R. 1993. Kinetic PCR: Real-time monitoring of DNA amplification reactions. Biotechnology 11: 1026-1030).

The following forward primer and reverse primer can be used as primer sequences for the purpose of detecting amplification of an amidase gene by a real-time PCR method.

Forward primer:
(SEQ ID NO: 3)
TGTCGCTCAATTAGCCAATGG
Reverse primer:
(SEQ ID NO: 4)
TGATGAGCCAGTGCAGCTCTT

The cycling protocol (cycle condition) for detecting amplification of an amidase gene by a real-time PCR method is as follows: at 50° C. for 2 minutes and 95° C. for 10 minutes, and 45 cycles at 95° C. for 15 seconds, and then at 60° C. for 60 seconds. The expression amount of an amidase gene of the self-cloning Aspergillus oryzae of the present invention is at least 2000 times or more, preferably 5000 times or more, and more preferably 10000 times or more, as compared to an original strain before self-cloning.

The self-cloning Aspergillus oryzae obtained by the present invention can be used in various industrial and commercial uses. For example, the self-cloning Aspergillus oryzae can be subjected to a contact treatment with an acrylamide-containing matter to reduce acrylamide from the acrylamide-containing matter. In addition, the self-cloning Aspergillus oryzae can be subjected to a contact treatment with an acrylamide-containing beverage and food to provide a method for producing a reduced-acrylamide beverage and food. Furthermore, an amidase protein can also be purified from the self-cloning Aspergillus oryzae to be added to an acrylamide-containing matter.

In the present invention, the “contact treatment” means that the self-cloning Aspergillus oryzae of the present invention is physically brought into contact with an acrylamide-containing matter. The acrylamide-containing matter is not restricted to a liquid but may be a solid or powder.

In the above-described contact treatment, the self-cloning Aspergillus oryzae of the present invention can be directly used, but can also be brought into contact with an acrylamide-containing matter in a state of having the self-cloning Aspergillus oryzae supported on a carrier (hereinafter also referred to as immobilized). As the carrier, appropriate materials such as dried gourd, cellulose, gel beads, porous glass beads, porous ceramics, and unwoven fabric can be used; however, a carrier with a coarse surface is preferable for adhesion of Aspergillus oryzae, and a porous carrier is preferable so that the self-cloning Aspergillus oryzae of the present invention does not weaken in culturing. Dried gourd can be prepared by a known method and, for example, can be prepared by drying gourd that is cut into an about 4 mm-square.

Known methods can be employed as a method of immobilization of the self-cloning Aspergillus oryzae of the present invention, and the self-cloning Aspergillus oryzae can be immobilized by, for example, a combination method and an entrapment method, but examples are not limited thereto. The combination method is a method of firmly fixing the self-cloning Aspergillus oryzae to a water insoluble carrier such as sintered glass, porous ceramics, porous glass beads, chitosan, celite, silica gel, zeolite, activated carbon, sponge, and cotton. The entrapment method is a method of taking a microbial cell body into a matrix made of a natural or synthesized polymer such as calcium alginate, polyethylene glycol, polyvinyl alcohol, polyurethane, polyacrylamide, carrageenan, agarose, cellulose, and dextrin.

In the above-described contact treatment, an acrylamide-containing matter and the self-cloning Aspergillus oryzae of the present invention can be brought into contact with each other under appropriate conditions. As a shaking method, known methods such as rotational shaking and reciprocal shaking can be employed, but examples are not limited thereto. In order to enhance a dissolved oxygen concentration during shaking, reciprocal shaking is preferable. The reciprocal shaking can be conducted with a lower limit of a rotational speed of 50 rpm or more, preferably 80 rpm or more, and more preferably 90 or more. The reciprocal shaking can be conducted with an upper limit of a rotational speed of 200 rpm or less, preferably 150 rpm or less, and more preferably 120 or less. When the rotational speed is 50 rpm or more, a microbial cell body adsorption amount is not in short, and when the rotational speed is 200 rpm or less, an amount of microbial cell bodies can be secured without violent contact of microbial cell bodies with one another. The lower limit of a temperature condition in shaking is 25° C. or higher, preferably 30° C. or higher, and more preferably 32° C. or higher. The upper limit of a temperature condition in shaking is 45° C. or lower, preferably 40° C. or lower, and more preferably 35° C. or lower. When the temperature condition in shaking is 25° C. or higher, the temperature does not lower than an optimal temperature of an enzyme reaction, and when the temperature is 45° C. or lower, deactivation of an enzyme does not occur.

The self-cloning Aspergillus oryzae of the present invention is subjected to a contact treatment with an acrylamide-containing beverage and food, thereby enabling production of a reduced-acrylamide beverage and food. Herein, the “beverage and food” indicates a beverage and a food, the “acrylamide-containing beverage and food” refers to one known as a beverage and food containing acrylamide, examples of the beverage include, but are not limited to, coffee, roasted green tea, green tea, black tea, oolong tea, bear and cacao beverages, and examples of the food include, but are not limited to, potato chips, fried potatoes, etc., which are processed products of potatoes, toast, cereal for breakfast, etc., which are grain processed products, chocolate products, dairy products, cocoa powder, biscuits for infants, and baby foods.

When a reduced-acrylamide beverage is produced, a liquid beverage is subjected to a contact treatment with the self-cloning Aspergillus oryzae of the present invention to reduce acrylamide. Then, the self-cloning Aspergillus oryzae is removed by a known isolation method such as precipitation or filtration, or can be inactivated by a known method such as high temperatures, low temperatures or freezing, but the methods are not limited thereto. In addition, when safety as an edible is approved, a reduced-acrylamide beverage can also be produced without removal and inactivation of the self-cloning Aspergillus oryzae.

When a reduced-acrylamide food is produced, in a production stage, the self-cloning Aspergillus oryzae of the present invention is mixed into raw materials of a food, or a liquid containing the self-cloning Aspergillus oryzae of the present invention can be sprayed to a solid food, but the method is not limited thereto. When the food is a liquid in the production stage, this liquid material can be subjected to a contact treatment with the self-cloning Aspergillus oryzae of the present invention. The liquid material subjected to the contact treatment with the self-cloning Aspergillus oryzae of the present invention can be processed into a solid product, or can be processed by rapidly freezing and drying the liquid material in a method such as a freeze dry treatment.

A reduced-acrylamide beverage and food also having reduced caffeine as compared to before the treatment can also be produced by the contact treatment with the self-cloning Aspergillus oryzae of the present invention.

A reduced-acrylamide beverage and food having reduced organic acids such as citric acid, malic acid, quinic acid, glycolic acid, lactic acid, formic acid and acetic acid as compared to before the treatment and having increased phosphoric acid as compared to before the treatment can also be produced by the contact treatment with the self-cloning Aspergillus oryzae of the present invention.

A reduced-acrylamide beverage and food having reduced chlorogenic acids such as monochlorogenic acid, feruloylquinic acid, and dicaffeoylquinic acid as compared to before the treatment can be produced by the contact treatment with the self-cloning Aspergillus oryzae of the present invention.

A reduced-acrylamide beverage and food having increased flavor components such as 1-propanol, ethyl acetate, 2-methyl-1-butanol, isobutyl alcohol and isoamyl alcohol as compared to before the treatment can be produced by the contact treatment with the self-cloning Aspergillus oryzae of the present invention. These flavor components can be increased twice, preferably 5 times, and more preferably 8 times as compared to before the treatment.

When the reduced-acrylamide beverage and food is coffee, various flavor components are increased or decreased and flowerlike fragrance is given to the coffee so that coffee having a light flavor without bitterness and thickness can be provided by the contact treatment with the self-cloning Aspergillus oryzae of the present invention.

### EXAMPLES

Next, the present invention will be more specifically described by way of production examples, test examples, and the like, but the invention is not limited by the examples described below.

### Production Example 1

### Preparation procedure of PenoA142

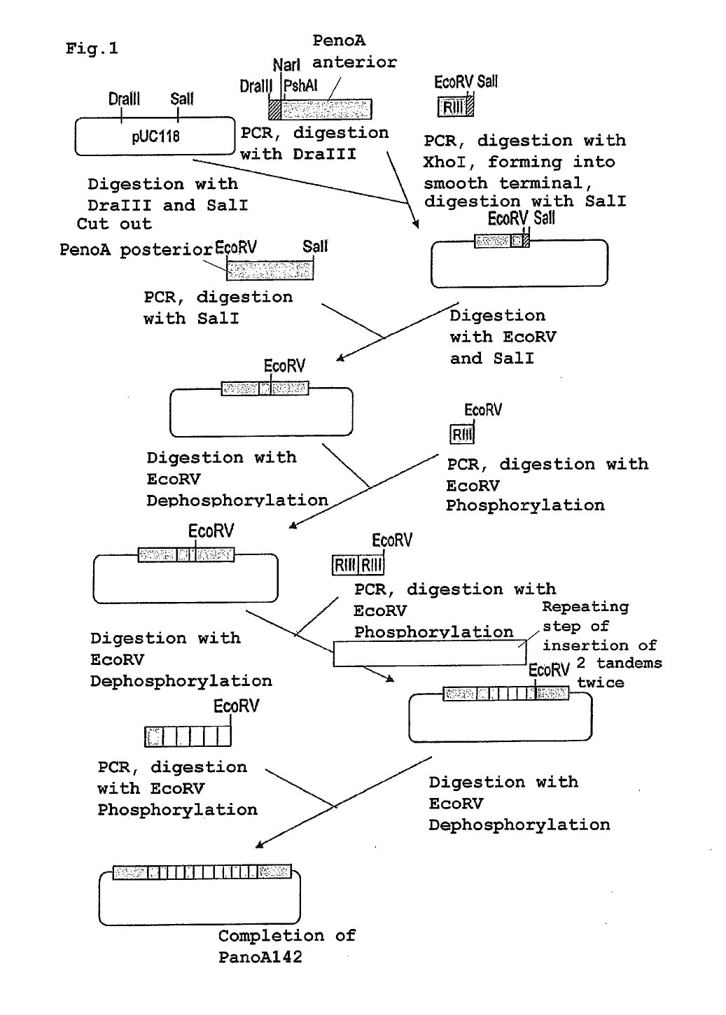

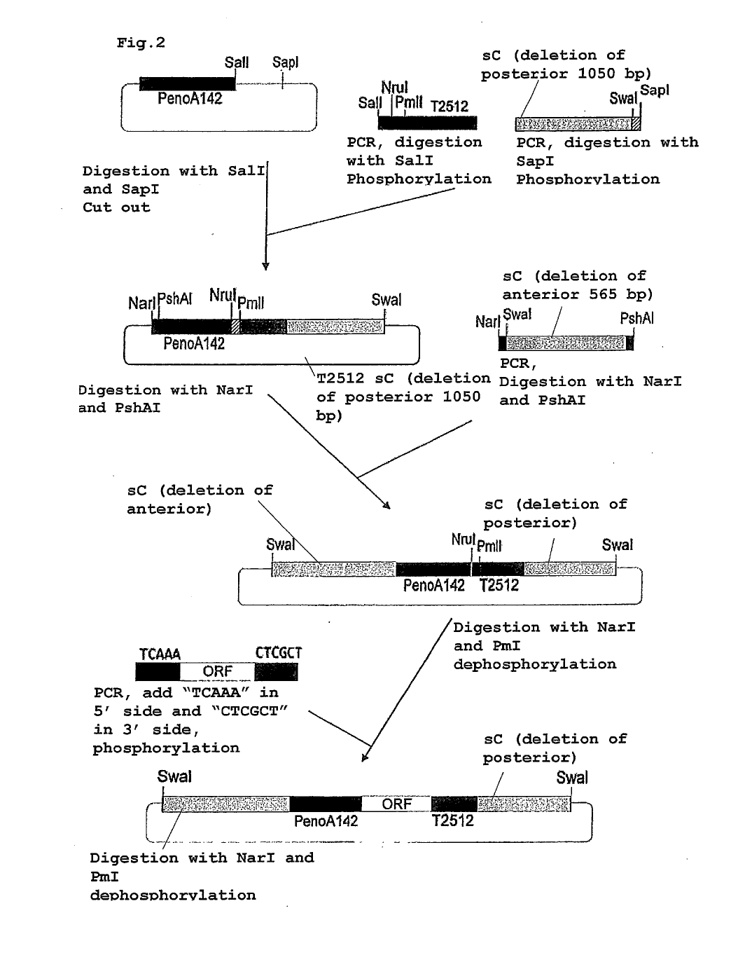

A preparation procedure of a pSENSelf2 plasmid will be described along with FIGS. 1 and 2. As shown in FIG. 1, a preparation procedure of PenoA142 that is a modified promoter derived from Aspergillus oryzae will be described. Plasmid pUC118 (manufactured by TAKARA BIO INC.) was digested with restriction enzymes DraIII and SalI. The anterior part of PenoA was amplified by a PCR method using an Aspergillus oryzae RIB40 strain genome (obtained from National Research Institute of Brewing) as a template by use of primers X1 (SEQ ID NO: 5) and Y1 (SEQ ID NO: 6), and digested with restriction enzyme DraIII; region III was further amplified by a PCR method using primers X2 (SEQ ID NO: 7) and Y2 (SEQ ID NO: 8), formed into a blunt end after the treatment with restriction enzyme XhoI and then digested with restriction enzyme SalI. Next, three fragments of the above-described pUC118, anterior part of PenoA and region III were ligated.

Then, the obtained plasmid was digested with restriction enzymes EcoRV and SalI. The posterior part of PenoA was amplified by a PCR method by use of primers X3 (SEQ ID NO: 9) and Y3 (SEQ ID NO: 10) and digested with restriction enzyme SalI, and two fragments were then ligated.

Subsequently, the obtained plasmid was digested with restriction enzyme EcoRV and dephosphorylated. The region III was further amplified by a PCR method using the Aspergillus oryzae RIB40 strain genome as a template by use of primers X4 (SEQ ID NO: 11) and Y4 (SEQ ID NO: 12), treated with restriction enzyme EcoRV and then phosphorylated. These two fragments were then ligated.

Subsequently, the obtained plasmid was digested with restriction enzyme EcoRV and dephosphorylated. Furthermore, the region III was amplified by a PCR method using the obtained plasmid as a template by use of the primers X4 (SEQ ID NO: 11) and Y4 (SEQ ID NO: 12), treated with restriction enzyme EcoRV and then phosphorylated (2 tandem fragments of the region III). These two fragments were ligated. Two of the region III 2 tandem fragments were further ligated to the plasmid obtained according to the above-mentioned method. This method was repeated twice to make the number of the region III become 6.

Six regions III were amplified by a PCR method using the obtained plasmid having 6 tandems of the regions III as a template by use of primers X5 (SEQ ID NO: 13) and Y4 (SEQ ID NO: 12), treated with restriction enzyme EcoRV and then phosphorylated. This fragment was introduced into a restriction enzyme EcoRV site of the same plasmid. Accordingly, PenoA142 (pUC118-PenoA142) was constructed.

### Production Example 2

### Construction of pSENSelf2 plasmid

As shown in FIG. 2, the above-described PenoA142 (pUC118-PenoA142) was digested with restriction enzymes SalI and SapI. A 2512 terminator was amplified by a PCR method using the Aspergillus oryzae RIB40 strain genome as a template by use of primers X6 (SEQ ID NO: 14) and Y6 (SEQ ID NO: 15), treated with restriction enzyme SalI and then phosphorylated. Next, an sC marker having deleted posterior 1050 bases was amplified by a PCR method using the Aspergillus oryzae RIB40 strain genome as a template by use of primers X7 (SEQ ID NO: 16) and Y7 (SEQ ID NO: 17), treated with restriction enzyme SapI and then phosphorylated. Subsequently, these three fragments were ligated.

The obtained plasmid was digested with restriction enzymes NarI and PshAI. Then, an sC marker having deleted anterior 565 bases was amplified by a PCR method using the Aspergillus oryzae RIB40 strain genome as a template by use of primers X8 (SEQ ID NO: 18) and Y8 (SEQ ID NO: 19), and treated with restriction enzymes NarI and PshAI. Subsequently, these two fragments were ligated.

Accordingly, a pSENSelf2 plasmid could be constructed. After transfection of an amidase gene into the pSENSelf2 plasmid, the pSENSelf2 plasmid is digested with restriction enzyme KpnI and a fragment having an amidase gene is purified so that a gene fragment only having Aspergillus oryzae derived sequences can be thus obtained. Performing transformation of Aspergillus oryzae by using this fragment made it possible to obtain a self-cloning strain having no foreign base.

### Production Example 3

### Preparation of pSENSelf2-amidase Plasmid

The obtained pSENself2 plasmid was digested with restriction enzymes PmlI and NruI, isolated and purified by agarose gel electrophoresis, and then dephosphorylated. The obtained plasmid was amplified by a PCR method using the Aspergillus oryzae RIB40 strain genome as a template by use of primers X9 (SEQ ID NO: 20) and Y9 (SEQ ID NO: 21), digested with restriction enzymes PmlI and NruI and phosphorylated. The primer X9 (SEQ ID NO: 20) contained 5 bases in the 3′ terminal of enoA 5′UTR and the primer Y9 (SEQ ID NO: 21) contained 6 bases in the 5′ terminal of the 2512 terminator, and these fragments were introduced by ligation. Accordingly, a pSENSelf2-amidase plasmid could be constructed.

### Production Example 4

### Preparation of Gene Fragment for Transformation

The obtained pSENself2-amidase plasmid was transfected into Escherichia coli DH5α, and the Escherichia coli was cultured with 50 mL of an LB medium containing 50 μg/mL of ampicillin sodium at 37° C. for overnight, and the Escherichia coli was recovered by centrifugation. A plasmid was purified and extracted from the Escherichia coli using a commercially available plasmid DNA purification kit (QIAprep Spin Miniprep Kit manufactured by Qiagen). This plasmid was then digested with restriction enzymes KpnI and SwaI, and about 7.5 kbp of a gene fragment for transformation, which was constituted with an sC marker and an amidase gene, was cut out to be purified by agarose gel electrophoresis.

### Production Example 5

### Preparation of Transformant and Confirmation by Southern Blotting Method

An Aspergillus oryzae NS4 strain (niaD and sC double deletion mutant strain derived from the RIB40 strain: subdivided from National Research Institute of Brewing) (reference: Biosci. Biotech. Biochem., 61(8), 1367-1369, 1997) was transformed using 10 μg of the obtained gene fragment for transformation by a protoplast PEG method (reference: Journal of The Society for Biotechnology, Vol. 76, No. 5, 187-193, 1998) to thus obtain 30 strains of transformants. These transformants were subjected to shaking culture with a dextrin and peptone medium (2% dextrin, 1% polypeptone, 0.5% KH₂PO₄, 0.05% MgSO₄.7H₂O) at 30° C. for 3 days to separate the culture solution and the microbial cell bodies.

The copy number of gene fragments for transformation inserted into each strain was confirmed using a ΔΔCT method (reference: Relative Quantitation Of Gene Expression: ABI PRISM 7700 Sequence Ditection System: User Bulletin #2: Rev B). That is, a real-time PCR was conducted using one copy of a gene that is present in a genome as a control and the copy number was presumed from the ratio. As this result, among 30 strains, there were 13 strains each having copy number 1 of inserting gene fragments for transformation, there were 9 strains each having copy number 2, there were 2 strains each having copy number 3, and there were 2 strains each having copy number 4. There were 4 strains into which gene fragments for transformation were not inserted.

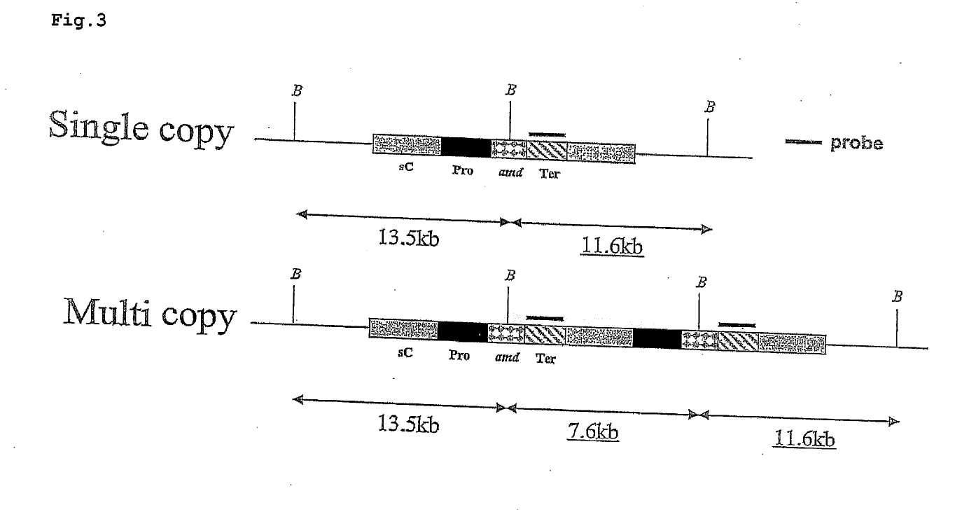

FIG. 3 shows a schematic view of gene fragments for transformation on a chromosome. The single copy (the copy number 1) in the upper part shows a state in which a gene fragment for transformation is incorporated into a chromosome of self-cloning Aspergillus oryzae. The multi copy in the lower part shows a state in which a plurality of gene fragments for transformation were incorporated into a chromosome of self-cloning Aspergillus oryzae (the copy number 2 in this figure).

Then, 4 strains were selected from the obtained transformants and a genome DNA was extracted by a general method. This genome DNA was digested with restriction enzyme BglII, and analyzed by a southern blotting method using a probe that recognizes a terminator region or a pUC118 region. In FIG. 3, lateral bars above the terminator sequence show recognition sites for a probe by the southern blotting method described below. FIG. 3 shows that 11.6 kbp of the genome DNA was formed in the single copy strain due to a restriction site by a restriction enzyme, which enzyme is shown by the italic letter B, and 11.6 kbp and 7.6 kbp of the genome DNA were formed in the multi copy. In addition, since BglII never cuts the pUC118 region of a vector, even if the pUC118 region is mixed in and is incorporated into a genome, it can be detected.

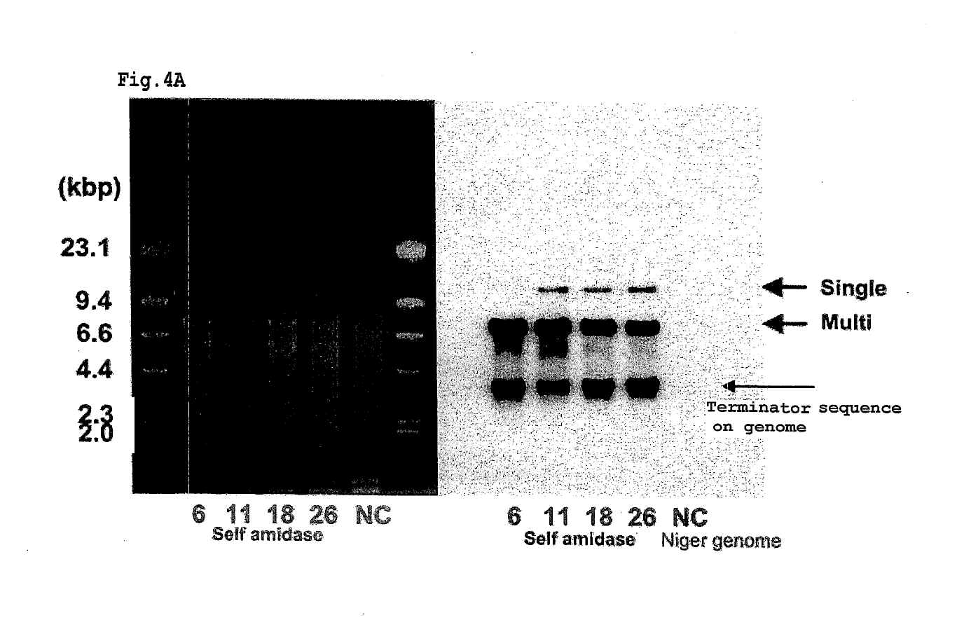

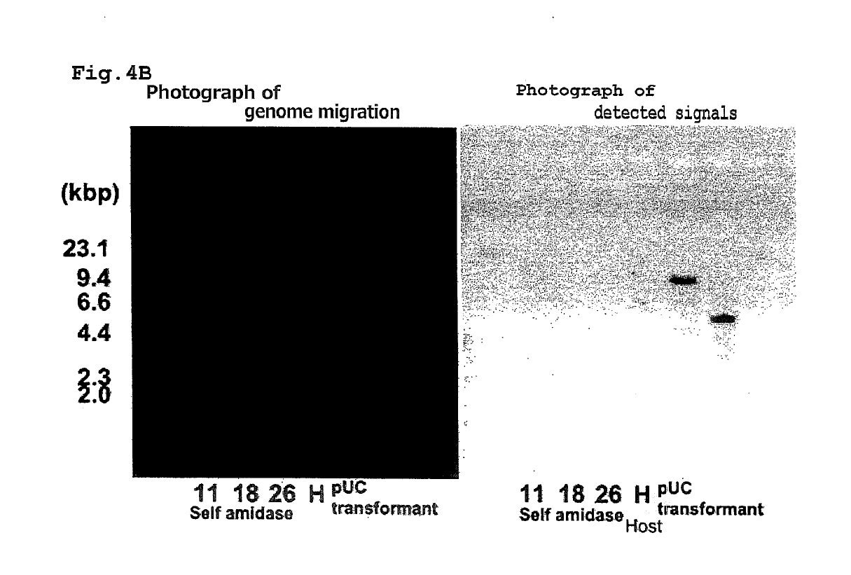

FIGS. 4A and 4B show analysis results by the southern blotting method. FIG. 4A shows analysis results in the case of using a probe that recognizes a terminator region, and FIG. 4B shows analysis results in the case of using a probe that recognizes a pUC118 region. As shown in FIG. 4A, two bands of 11.6 kbp and 7.6 kbp were detected in the sample Nos. 11, 18 and 26, and insertion of gene fragments for transformation into a host chromosome in a homologous state was confirmed. The band of 7.6 kbp was detected but that of 11.6 kbp was not detected in the sample No. 6; therefore, insertion of gene fragments for transformation into a host chromosome in a heterologous state was confirmed. In addition, the band of 3.7 kbp is derived from a terminator that is present in a host genome. As shown in FIG. 4B, when a probe that recognizes the pUC118 region was used, no band was detected in the samples 11, 18 and 26. From the analysis results, it was confirmed that an Escherichia coli plasmid-derived DNA is not inserted into the genome of the transformant and the transformant is therefore a self-cloning strain.

### Test Example 1

### Measurement of Amidase Specific Ratio of Self-Cloning Aspergillus oryzae

An NS4 strain that is an original strain before conducting self-cloning (also referred to as a parent strain), an Aspergillus oryzae strain having copy number 1, an Aspergillus oryzae strain having copy number 2, an Aspergillus oryzae strain having copy number 3, and an Aspergillus oryzae strain having copy number 4 were each subjected to shaking culture using a YPD (Yeast peptone dextrose) medium (yeast extract 1%, peptone 2%, dextrose 2%, all expressed by w/v, pH 6.5) with 2×10⁷ spores/mL at 30° C. and 100 rpm for 3 days. A No. 100 strain was subjected to shaking culture using a YPD medium at 30° C. and 100 rpm for 3 days and then to shaking culture using a 200 ppm-acrylamide-added CD (Czapek-Dox) medium (sucrose 3%, NaNO₃ 0.3%, MgSO₄.7H₂O 0.05%, KCl 0.05%, K₂HPO₄ 0.01%, FeSO₄.H₂O 0.001%, all expressed by w/v, pH 9.0) at 35° C. and 100 rpm for 2 days. Herein, the No. 100 strain is not a self-cloning strain but a strain which has been known to produce a larger amount of amidase than a conventional strain does by conducting induction culture with an acrylamide-added CD medium (Patent Document 1).

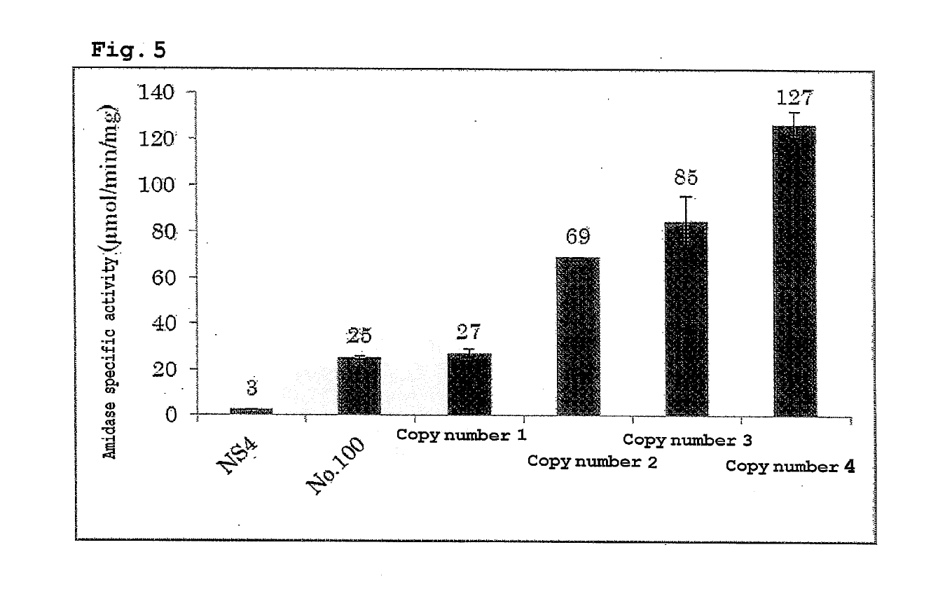

FIG. 5 shows measurement results of amidase specific activity. The vertical axis in FIG. 5 shows measured amidase specific activity values (μmol/min/mg), and the horizontal axis shows species of strains that were used in the experiment. Aspergillus oryzae spores were subjected to shaking culture using a YPD medium with 1×10⁷ spores/mL at 30° C. and 100 rpm for 3 days, microbial cell bodies were then collected and washed, the microbial cell bodies frozen by liquid nitrogen were then ground with a mortar, and thereto was added 0.4 mL of a 0.1 M-Mcllvaine buffer solution (pH 7.0) which was a twice amount of the amount of the wet microbial cell bodies to thus extract an enzyme in the microbial cell bodies. To 0.4 mL of the obtained extraction solution was added 0.4 mL of a 0.1 M-Mcllvaine buffer solution containing 2000 ppm acrylamide (pH 7.0), and the resultant was reacted at 30° C. for 30 minutes, and then thereto was added 0.2 mL of 0.5 N—HCl to terminate the reaction.

This reaction solution was filtered with a 0.45 μm-membrane filter and an amount of a generated acrylic acid is quantitatively determined by HPLC. A specific activity of amidase was expressed by an acrylic acid amount generated in 1 minute per 1 mg of a protein. For acrylamide, one manufactured by Tokyo Chemical Industry Co., Ltd. was used. LC-2010AHT HPLC system manufactured by SHIMADZU CORPORATION was used as HPLC and CAPCELL PAK C8 manufactured by Shiseido Company, Limited was used as the column. A mobile phase was a 0.1% (w/v) aqueous phosphoric acid solution and the measurement was conducted under the measurement conditions of a column temperature of 40° C., a detection wavelength of 200 nm and a solution sending speed of 1 mL/minute.

### Test Example 2

### Measurement of Expression Amount of Amidase Gene in Self-Cloning Aspergillus oryzae

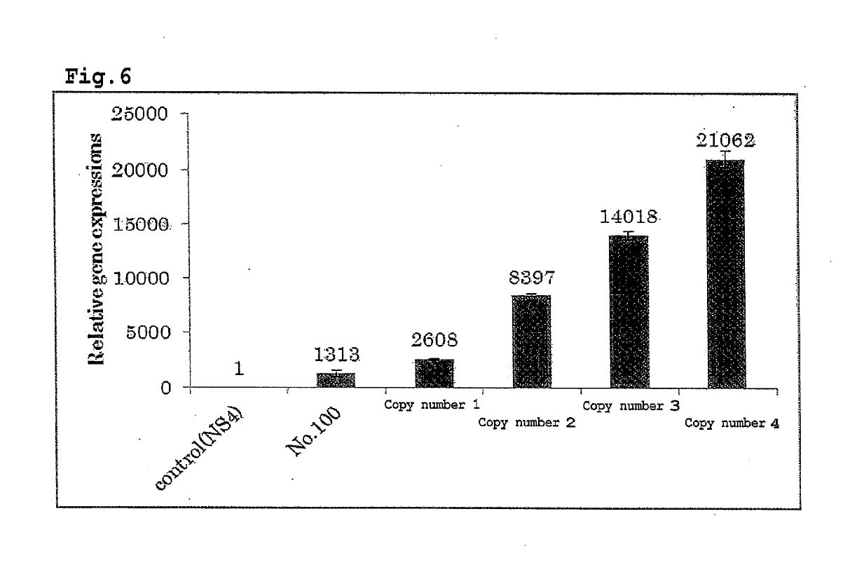

FIG. 6 shows results of measuring the expression amount of an amidase gene in self-cloning Aspergillus oryzae by a real-time PCR method. The vertical axis in FIG. 6 shows the expression amounts of amidase genes in respective Aspergillus oryzae strains assuming an original strain (NS4) without self-cloning as 1. The horizontal axis shows species of Aspergillus oryzae strains used in the experiment. A 7500 Real-Time PCR System (manufactured by Applied Biosystems) was used for the real-time PCR equipment, and the following forward primer and reverse primer were used as primer sequences in order to detect amplification of amidase genes.

Forward primer:
(SEQ ID NO: 3)
TGTCGCTCAATTAGCCAATGG
Reverse primer:
(SEQ ID NO: 4)
TGATGAGCCAGTGCAGCTCTT

The cycling protocols to detect amplification of amidase genes were as follows: at 50° C. for 2 minutes and 95° C. for 10 minutes, and 45 cycles at 95° C. for 15 seconds, and then at 60° C. for 60 seconds.

### Test Example 3

### Test of Acrylamide Reduction in Acrylamide Added Water

An NS4 strain that is an original strain before conducting self-cloning, an Aspergillus oryzae strain having copy number 1, an Aspergillus oryzae strain having copy number 2, an Aspergillus oryzae strain having copy number 3, and an Aspergillus oryzae strain having copy number 4 were each subjected to shaking culture using a YPD (Yeast peptone dextrose) medium (yeast extract 1%, peptone 2%, dextrose 2%, all expressed by w/v, pH 6.5) at 30° C. and 100 rpm for 3 days.

Dried gourd was prepared by cutting commercially available gourd into an about 4 mm-square, adsorbing 1.5 mL of the YPD medium thereonto as the source of nutrition, sterilizing in an autoclave, and then drying at 60° C. for 24 hours. To a 100 mL-container were added 0.5 g of the dried gourd and 40 mL of the YPD medium, and the resultant was sterilized in an autoclave, each self-cloning Aspergillus oryzae was then inoculated with 2×10⁷ spores/mL, and thereafter the resultant was subjected to shaking culture at 30° C. and 100 μm for 3 days to immobilize each strain to the dried gourd. This immobilized strain was washed with sterilized water. The No. 100 strain was subjected to shaking culture at 30° C. and 100 μm for 3 days using a 200 ppm-acrylamide-added CD medium in order to induce production of amidase. Herein, the dried gourd is easily attached with microbial cell bodies and suitable for nurture of microbial cell bodies. The microbial cell bodies can be maintained in the dried gourd without damaging the microbial cell bodies even when shaking culture is conducted, and therefore the dried gourd in which the microbial cell bodies were immobilized also can be reused.

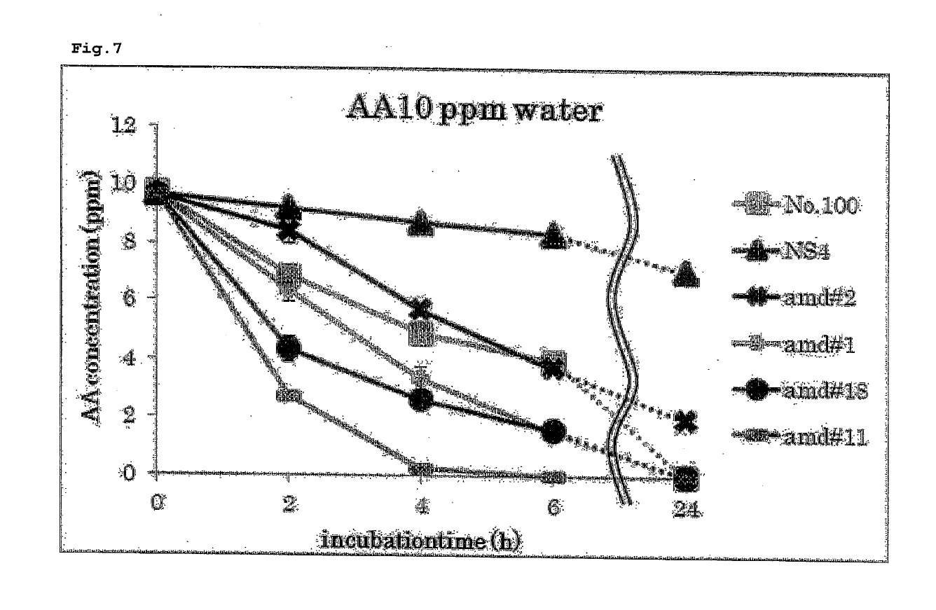

The above-described immobilized strain was added to 10 ppm acrylamide-added water and a reaction was initiated by reciprocal shaking at 35° C. and 100 rpm. The reaction solution was recovered after the initiation of the reaction of 0 hours, 2 hours, 4 hours, 6 hours and 24 hours and filtered with a 0.45 μm-filter, and the concentration of acrylamide was then measured by HPLC. FIG. 7 shows results of a test of acrylamide reduction in 10 ppm acrylamide-added water. The vertical axis in FIG. 7 shows a residual ratio (%) of acrylamide, and the horizontal axis shows an elapsed time of a contact treatment to each self-cloning Aspergillus oryzae. In FIG. 7, the No. 100 strain is not a self-cloning strain but a strain which has been known to produce a larger amount of amidase than a conventional strain does by conducting induction culture with a 200 ppm acrylamide-added CD medium, and the NS4 strain is an original strain before conducting self-cloning. In addition, the sample number amd #2 shows a self-cloning Aspergillus oryzae strain having copy number 1, the sample number amd #1 shows a self-cloning Aspergillus oryzae strain having copy number 2, the sample number amd #18 shows a self-cloning Aspergillus oryzae strain having copy number 3, and the sample number amd #11 shows a self-cloning Aspergillus oryzae strain having copy number 4.

Herein, a reaction by reciprocal shaking is effective since efficiency of acrylamide reduction can be enhanced by increasing the dissolved oxygen in the reaction solution. Herein, setting the reaction temperature at 35° C. is effective since the temperature is an optimal temperature for degradation by the enzyme and efficiency of acrylamide reduction can be thus enhanced.

### Test Example 4

### Test of Acrylamide Reduction in Acrylamide-Added Coffee

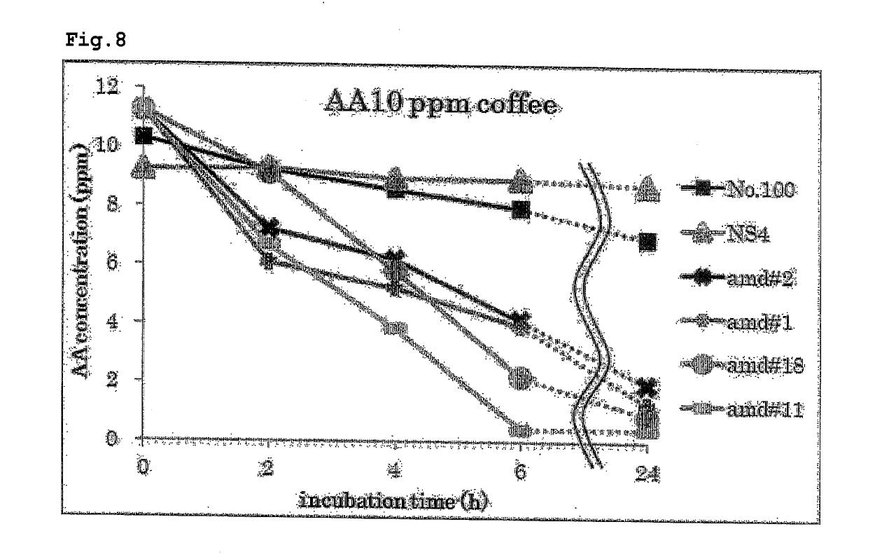

The above-described immobilized strain was added to 10 ppm acrylamide-added coffee and a reaction was initiated by reciprocal shaking at 35° C. and 100 rpm. The reaction solution was recovered after the initiation of the reaction of 0 hours, 2 hours, 4 hours, 6 hours and 24 hours and filtered with a 0.45 μm-filter, and the concentration of acrylamide was then measured by HPLC. FIG. 8 shows results of a test of acrylamide reduction in 10 ppm acrylamide-added coffee. The vertical axis shows a residual ratio (%) of acrylamide, and the horizontal axis shows an elapsed time of a contact treatment to each self-cloning Aspergillus oryzae. In FIG. 8, a sample number of each Aspergillus oryzae is the same as that in Test Example 3.

### Test Example 5

### Test of Acrylamide Reduction in Acrylamide-Free Coffee

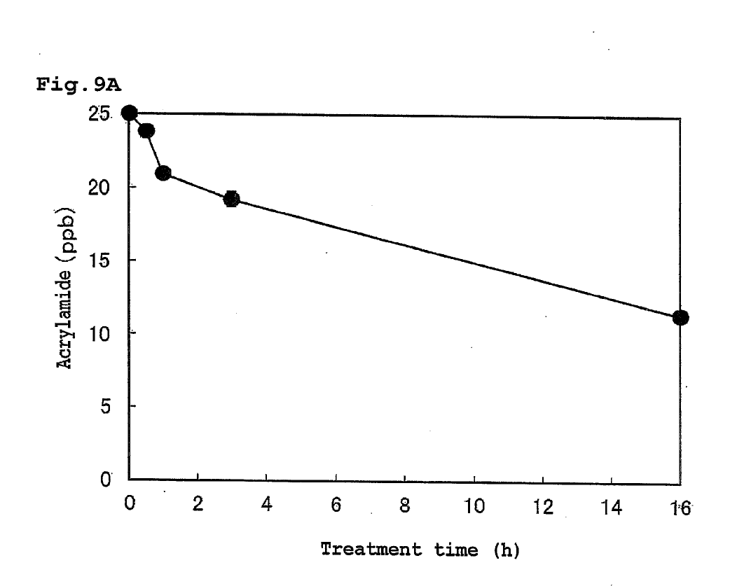

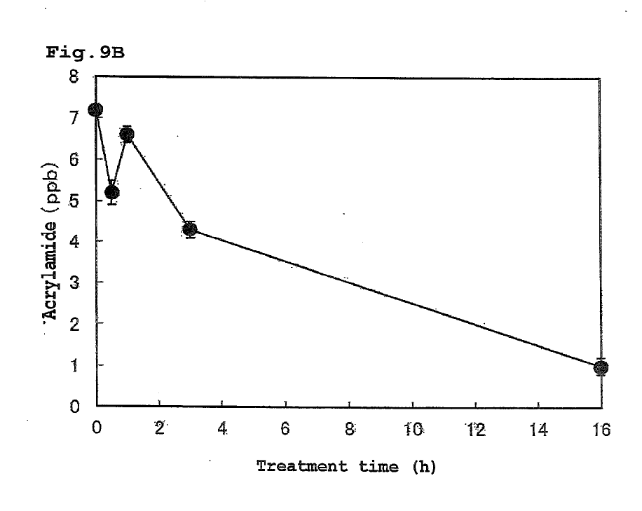

FIG. 9A shows results of measuring an effect of acrylamide reduction in an acrylamide-free coffee extraction solution by GC-MS. The vertical axis in FIG. 9A shows a residual amount of acrylamide (ppb), and the horizontal axis shows an elapsed time of a contact treatment to the self-cloning Aspergillus oryzae of the present invention. After crushing coffee beans (L value of 17.7), hot water at 95° C. was added to the coffee beans to have a water addition ratio of 1:17, followed by extraction, and the resultant was then naturally cooled to room temperature to yield a coffee extraction solution. The above-described immobilized strain (Aspergillus oryzae strain having copy number 4) was added to this coffee extraction solution, and the resultant was reacted by reciprocal shaking at 35° C. and 100 rpm. Solid-phase extraction was conducted on this coffee extraction solution as a pretreatment to remove contaminants in the sample.

Then, the coffee extraction solution was subjected to derivatization and then to GC-MS (GCMS-QP2010, manufactured by SHIMADZU CORPORATION). As the GC conditions, a ZB-1 column (30 m×0.32 mm I.D., manufactured by SHIMADZU GLC Ltd.) with a film thickness of 1.0 μm was used, the column temperature was at 70° C. for 1 minutes, increased to 120° C. at 12° C./minute, increased from 120° C. to 160° C. at 5° C./minute, and then increased at 20° C./minute and set at 300° C. for 5 minutes. The vaporization chamber temperature was 270° C. and helium gas was used as a carrier gas. GC was conducted at a linear velocity of 55 cm/second. As the MS conditions, an ion source was 270° C., a detector voltage was 0.05 kv, a SIM samplate was 0.2 seconds, and acrylamide, acrylamide 13C3, naphthalene-d8 and phenanthrene were used for selected ions.

FIG. 9B shows results of measuring an effect of acrylamide reduction in an acrylamide-free coffee product by GC-MS. The vertical axis in FIG. 9B shows a residual amount of acrylamide (ppb), and the horizontal axis shows an elapsed time of a contact treatment to Aspergillus oryzae. A content liquid in a canned coffee that is manufactured by general production processes and sold (sugar-free type: BRIX 1.0) was used as the coffee product. The above-described immobilized strain (Aspergillus oryzae strain having copy number 4) was added to this canned coffee, and the resultant was reacted by reciprocal shaking at 35° C. and 100 rpm. Thereafter, the canned coffee was subjected to derivatization and then to GC-MS (GCMS-QP2010, manufactured by SHIMADZU CORPORATION) under the above-described conditions.

### Test Example 6

### Test of Caffeine Reduction in Coffee Extraction Solution

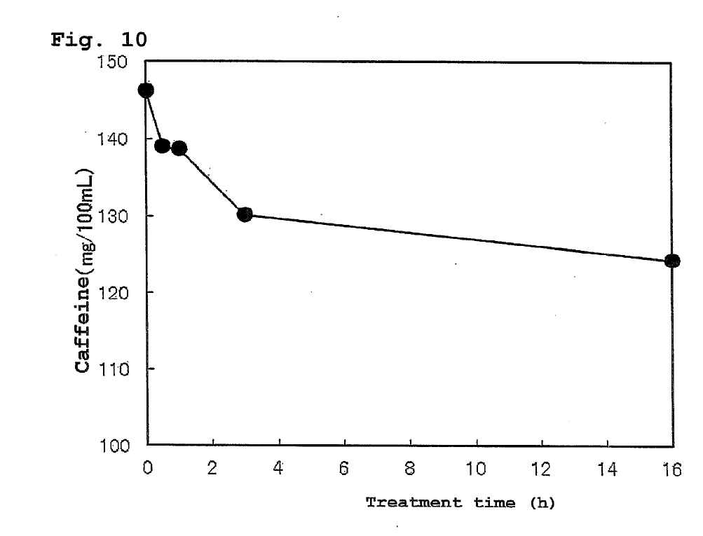

FIG. 10 shows results of a measurement of the chronological change in caffeine content in the above-described coffee extraction solution. The vertical axis in FIG. 10 shows a caffeine amount (mg/100 mL), and the horizontal axis shows a treatment time (hour). The preparation of the coffee extraction solution, the contact with the immobilized strain (Aspergillus oryzae strain having copy number 4) and the reaction were conducted according to the method described in Test Example 5. The calibration curve was created by determining an area ratio of caffeine and β-phenethyl alcohol, which was obtained from HPLC chromatography, using β-phenethyl alcohol as the internal standard. The caffeine in the coffee extraction solution was analyzed by HPLC and an area ratio of the caffeine and β-phenethyl alcohol was determined to calculate a caffeine content in a sample. The measurement was conducted using Nucleosil 10 C18 (250 mm×4 mm I.D.) for the HPLC column and a solution obtained by mixing methanol and 0.2 M-perchloric acid at a ratio of 2:8 for the mobile phase at a flow rate of 1.0 mL/minute. An ultraviolet spectrophotometer (detection wavelength of 270 nm) was used for detection.

### Test Example 7

### Measurement Results of Amounts of Phosphoric Acid and Organic Acids in Coffee Extraction Solution

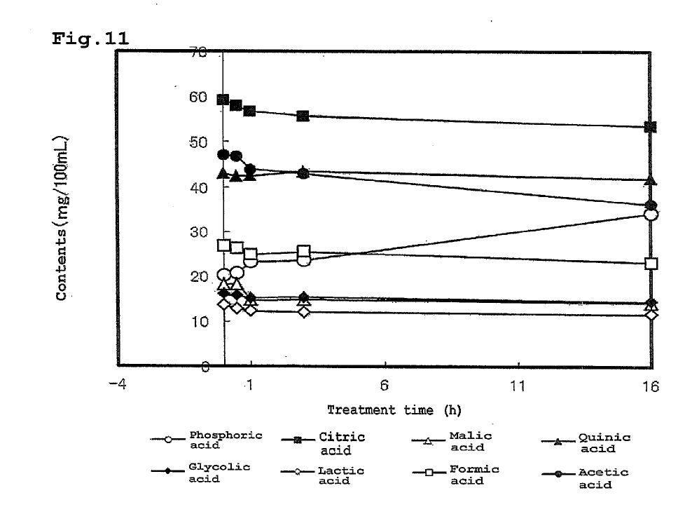

FIG. 11 shows results of conducting a quantitative analysis of phosphoric acid and organic acids (citric acid, malic acid, quinic acid, glycolic acid, lactic acid, formic acid and acetic acid) in a coffee extraction solution in a post-labeling (BTB indicator) detection method by HPLC. The vertical axis in FIG. 11 shows contents (mg/100 mL) of phosphoric acid and organic acids in the coffee extraction solution, and the horizontal axis shows a time for a contact treatment between self-cloning Aspergillus oryzae and the coffee extraction solution. The preparation of the coffee extraction solution, the contact with the immobilized strain (Aspergillus oryzae strain having copy number 4) and the reaction were conducted according to the method described in Test Example 5. The measurement was conducted using Shodex RSpak KC-811 (30 cm×8 mm I.D.×4) for the columns at a column temperature of 60° C., and 3 mM HClO₄/H₂O for the mobile phase at a flow rate of 1 mL/minute. The measurement was conducted using 15 mM Na₂HPO₄, 2 mM NaOH and 0.2 mM BTB for a labeling solution at a flow rate of 0.5 mL/minute. An ultraviolet spectrophotometer (detection wavelength of 445 nm) was used for detection.

### Test Example 8

### Measurement Results of Amount of Chlorogenic Acids in Coffee Extraction Solution

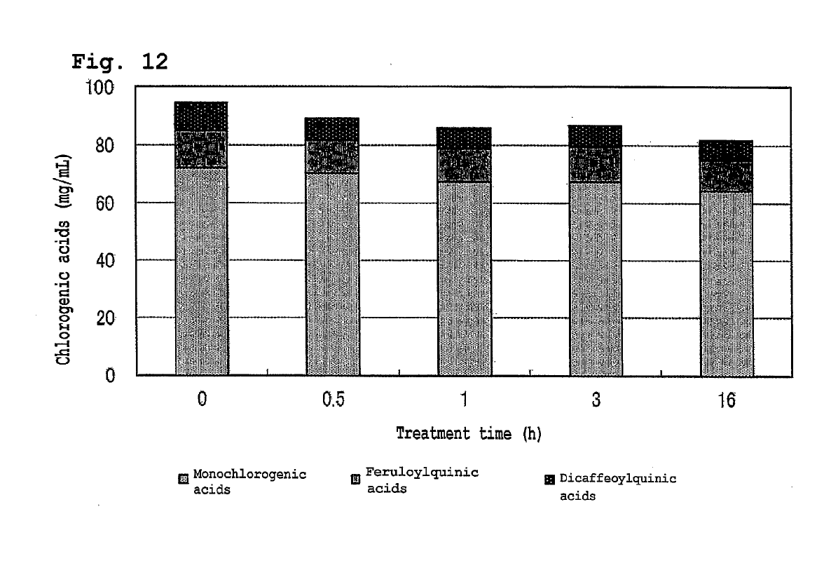

FIG. 12 shows results of conducting a quantitative analysis of chlorogenic acids (monochlorogenic acids, feruloyl quinic acids and dicaffeoyl quinic acids) in a coffee extraction solution by HPLC. The vertical axis in FIG. 12 shows contents (mg/mL) of chlorogenic acids in the coffee extraction solution, and the horizontal axis shows a time for a contact treatment between self-cloning Aspergillus oryzae and the coffee extraction solution. The preparation of the coffee extraction solution, the contact with the immobilized strain (Aspergillus oryzae strain having copy number 4) and the reaction were conducted according to the method described in Test Example 5. The measurement was conducted using Inertsil ODS-3 (150 mm×4.6 mm I.D.) for the column at a column temperature of 40° C., and A) a 10 mM phosphoric acid buffer solution and B) a solution of 10 mM phosphoric acid in acetonitrile for the mobile phase at a flow rate of 1 mL/minute. Table 1 shows gradient conditions during HPLC.

### (Gradient Conditions During HPLC)

TABLE 1

| min | A (%) | B (%) |
|---|---|---|
| 0 | 95 | 5 |
| 30 | 80 | 20 |
| 45 | 65 | 35 |
| 50 | 20 | 80 |
| 65 | 95 | 5 |

### Test Example 9

### Change of Flavor Components in Coffee Extraction Solution

Table 2 shows results of measuring variation of flavor components in a coffee extraction solution due to a contact treatment with self-cloning Aspergillus oryzae by GC-MS. Flavor components among Table 2 which were enhanced by contact treatment are shown in Table 3. The preparation of the coffee extraction solution, the contact with the immobilized strain (Aspergillus oryzae strain having copy number 4) and the reaction were conducted according to the method described in Test Example 5. The measurement was conducted under the headspace conditions of a temperature of 60° C., a retention time of 30 minutes, a transfer temperature of 180° C., a needle temperature of 120° C., a sample injection time of 0.1 minutes, and a carrier gas pressure of 110 kPa. The measurement was conducted under the GC conditions of using column ZB (10.32 mm I.D.), a film thickness of 3.0 μm, a column temperatures of 40° C. (5 minutes)−5° C./minute−60° C.−15° C./minute−250° C. (3 minutes), a He pressure of 80 kPa, an injection port temperature of 250° C., a split ratio of 0, and a split flow amount of 20.4 mL/minute. The measurement was conducted under the MS conditions of an interface temperature of 300° C. and a SIM sampling rate of 0.2 seconds.

TABLE 2

| | | | Increase (↑), | m/z peak area | | | | | |
|---|---|---|---|---|---|---|---|---|---|
| | | m/z peak | decrease (↓), | | After | | | | |
| | | area ratio | small change | After 0 | 0.5 | After 1 | After 3 | After 16 | |
| No. | Volatile components | 16 h/0 h | (→) | hours | hours | hour | hours | hours | m/z |
| 1 | Acetaldehyde | 0.3 | ↓ | 1434623 | 1707346 | 1480435 | 709449 | 405129 | 29 |
| 2 | Methyl formate | 0.6 | ↓ | 246591 | 239554 | 231580 | 224432 | 140599 | 31 |
| 3 | Ethanol | 2.7 | ↑ | 2533695 | 3781351 | 3424802 | 3183329 | 6847548 | 31 |
| 4 | Acetone | 0.9 | → | 2279507 | 2340945 | 2233330 | 2200843 | 2047409 | 43 |
| 5 | Propanal | 0.0 | ↓ | 350218 | 281067 | 29932 | 9925 | 4884 | 29 |
| 6 | Furan | 0.1 | ↓ | 339280 | 234919 | 215803 | 220077 | 21529 | 39 |
| 7 | Methyl acetate | 0.5 | ↓ | 1358628 | 1351196 | 1258531 | 1063540 | 718366 | 43 |
| 8 | Isobutyl aldehyde | 0.1 | ↓ | 924272 | 912127 | 742092 | 286292 | 63757 | 43 |
| 9 | 1-Propanol | 15.5 | ↑ | 11518 | 68659 | 155941 | 163650 | 178191 | 31 |
| 10 | Acetic acid | 0.6 | ↓ | 49286 | 48890 | 44654 | 42512 | 28139 | 43 |
| 11 | Diacetyl | 0.1 | ↓ | 603368 | 676888 | 555149 | 312648 | 60716 | 43 |
| 12 | Butanal | 0.0 | ↓ | 39134 | 34182 | 1276 | 0 | 0 | 44 |
| 13 | 2-Butanone | 0.9 | → | 824585 | 830929 | 820797 | 800106 | 736427 | 43 |
| 14 | 2-Methylfuran | 0.0 | ↓ | 1267776 | 825793 | 768596 | 714960 | 39127 | 82 |
| 15 | Ethyl acetate | 9.0 | ↑ | 10152 | 24020 | 20964 | 28376 | 91598 | 43 |
| 16 | 3-Methylfuran | 0.0 | ↓ | 56170 | 37138 | 35384 | 33788 | 2265 | 82 |
| 17 | Isobutyl alcohol | 7.3 | ↑ | 21150 | 25074 | 42326 | 76288 | 154267 | 43 |
| 18 | Methyl propionate | 0.2 | ↓ | 32736 | 30363 | 27168 | 19183 | 7131 | 57 |
| 19 | Isovaleraldehyde | 0.2 | ↓ | 553508 | 543456 | 454879 | 75877 | 121471 | 41 |
| 20 | 1-Butanol | 0.4 | ↓ | 114459 | 108621 | 26725 | 46241 | 47265 | 56 |
| 21 | 2-Methylbutylaldehyde | 0.0 | ↓ | 1063848 | 1021982 | 674879 | 127947 | 42978 | 57 |
| 22 | Thiophene | 0.2 | ↓ | 42422 | 32832 | 32290 | 29863 | 6445 | 84 |
| 23 | 2-Pentanone | 2.2 | ↑ | 95996 | 97546 | 107115 | 164165 | 215055 | 43 |
| 24 | 2,3-Pentanedione | 0.0 | ↓ | 388263 | 417299 | 328205 | 149767 | 13392 | 43 |
| 25 | 3-Pentanone | 0.8 | ↓ | 105857 | 102190 | 98247 | 98274 | 86857 | 57 |
| 26 | 2,5-Dimethylfuran | 0.1 | ↓ | 89135 | 57976 | 50167 | 43730 | 5246 | 96 |
| 27 | Pyrazine | 0.9 | → | 49203 | 47911 | 48187 | 47243 | 45683 | 80 |
| 28 | Isoamyl alcohol | 6.2 | ↑ | 35202 | 39592 | 59317 | 149475 | 217729 | 55 |
| 29 | 2-Methyl-1-butanol | 8.7 | ↑ | 14359 | 14625 | 36162 | 89219 | 124435 | 57 |
| 30 | 1-Methylpyrrole | 0.4 | ↓ | 78593 | 65697 | 67198 | 60992 | 34310 | 81 |
| 31 | Pyridine | 1.2 | ↑ | 340812 | 369566 | 379580 | 365635 | 411132 | 79 |
| 32 | Dimethyl disulfide | 0.3 | ↓ | 46157 | 40681 | 37492 | 36227 | 11878 | 94 |
| 33 | 4-Methyl-2,3-pentanedione | 0.1 | ↓ | 87820 | 94838 | 62513 | 21840 | 5811 | 43 |
| 34 | 3-Hexanone | 0.8 | ↓ | 22556 | 20686 | 19531 | 20032 | 17505 | 43 |
| 35 | Dihydro-2-methyl-3-furanone | 0.9 | → | 112706 | 119844 | 107971 | 103467 | 102830 | 43 |
| 36 | 2-Methylpyrazine | 1.0 | → | 268280 | 282640 | 264157 | 262771 | 260681 | 94 |
| 37 | Furfural | 0.0 | ↓ | 142164 | 159546 | 187932 | 129237 | 1182 | 96 |
| 38 | 2-Furfurylmethylether | 0.9 | → | 60973 | 59884 | 58428 | 59532 | 53809 | 81 |
| 39 | Acetoxy 2-propanone | 0.6 | ↓ | 120346 | 120860 | 111198 | 103319 | 72387 | 43 |
| 40 | Furfuryl alcohol | 1.0 | → | 326104 | 332318 | 311285 | 324136 | 328594 | 98 |
| 41 | Acetylfuran | 0.9 | → | 87508 | 86158 | 85432 | 65205 | 79823 | 95 |
| 42 | 2,6-Dimethylpyrazine | 1.0 | → | 210075 | 214845 | 205630 | 204103 | 207648 | 108 |
| 43 | 2-Ethylpyrazine | 1.0 | → | 84185 | 88548 | 86406 | 84500 | 83734 | 107 |
| 44 | 5-Methylfurfural | 0.0 | ↓ | 115121 | 121083 | 102452 | 62532 | 1836 | 110 |
| 45 | Furfuryl acetate | 0.1 | ↓ | 374629 | 362768 | 293133 | 184807 | 37633 | 81 |
| 46 | 2-Ethyl-6-methylpyrazine | 1.0 | → | 80878 | 78717 | 73627 | 79244 | 80010 | 121 |
| 47 | 2-Ethyl-3-methylpyrazine | 1.0 | → | 41380 | 48172 | 43498 | 46467 | 42381 | 121 |
| 48 | Furfuryl propionate | 0.2 | ↓ | 31016 | 25516 | 20232 | 10905 | 5868 | 81 |
| 49 | 2-Ethyl-3,6-dimethylpyrazine | 1.1 | → | 55691 | 60219 | 57006 | 57253 | 59260 | 135 |
| 50 | Furfurylpyrrole | 0.7 | ↓ | 55248 | 49075 | 48138 | 44275 | 39736 | 81 |

TABLE 3

| | | Increase ratios of flavor |
|---|---|---|
| | Characteristics of | components after 16 hours |
| Components | fragrances | (based on 0 hours as 1) |
| 1-Propanol | Sweet and comfortable | 15.5 |
| | fragrance expressed as an | |
| | alcoholic odor | |
| Ethyl acetate | Fruity flavor | 9.0 |
| | (the most general among | |
| | fruity flavors) | |
| 2-Methyl-1- | Fruity, wine-like flavor, | 8.7 |
| butanol | pungent | |
| Isobutyl alcohol | Sweet fragrance, one of | 7.3 |
| | fusel oils | |
| Isoamyl alcohol | One of fusel oils, highly | 6.2 |
| | concentrated, wine-like | |
| | and fruity flavor | |
| Ethanol | Ethanol odor | 2.7 |
| 2-Pentanone | Strong fruity and ethanol | 2.2 |
| | odor | |
| Pyridine | Index about bitterness | 1.2 |

### Test Example 10

### Sensory Evaluation Test

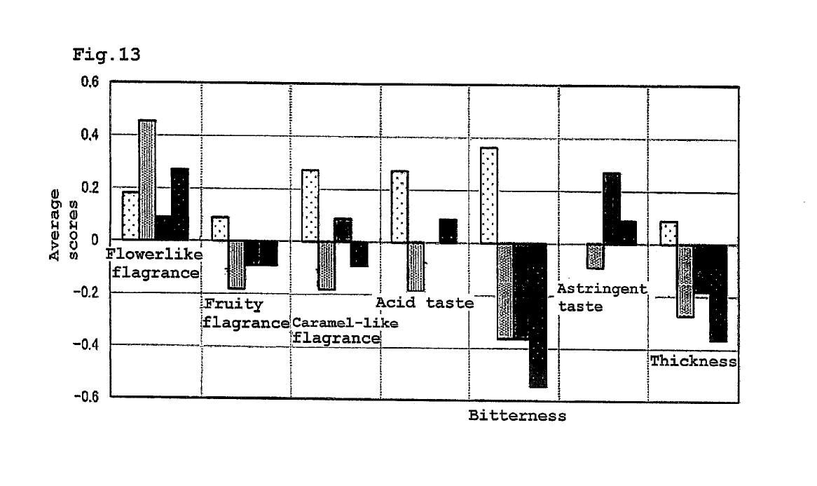

FIG. 13 shows results of a sensory evaluation test regarding change of flavors of coffee due to a contact treatment with self-cloning Aspergillus oryzae. The vertical axis in FIG. 13 shows an average score regarding each evaluation item, and the horizontal axis shows each evaluation item. Four kinds of bar graphs are shown for each evaluation item and indicate evaluation results before contact treatment, 3 hours after contact treatment, 6 hours after contact treatment, and 16 hours after contact treatment in the order from the left side.

Totally 11 people of 7 males and 4 females (average age of 30.5 years old) were selected from workers in their twenties to forties who belong to the R&D center in UCC UESHIMA COFFEE CO., LTD. as panelists, and the sensory evaluation test was performed. The preparation of the coffee extraction solution, the contact with the immobilized strain (Aspergillus oryzae strain having copy number 4) and the reaction were conducted according to the method described in Test Example 5. The coffee extraction solution sample and an untreated sample (control) were appropriately dispensed into plastic containers, and sample names were encoded and subjected at an initial temperature of 10° C., to perform the sensory evaluation test in a sensory examination room. The coffee extraction solution sample was in accordance with the preparation conditions of acrylamide-free coffee in Test Example 5.

As the quality of fragrance, “flowerlike fragrance”, “fruity fragrance” and “caramel-like fragrance” and, as the evaluations of tastes, “acid taste”, “bitterness”, “astringent taste”, “thickness” and “after taste” were selected as evaluation items, in reference to terms for attribute evaluation which were suggested by Hayakawa et al. (reference: Hayakawa, F., Kazami, Y., Wakayama, H., Oboshi, R., Tanaka, H., Maeda, G., Hoshino, C., Iwawaki, H and Iyabayashi, T. Sensory Lexicon of Brewed Coffee for Japanese Consumers, Untrained Coffee Professionals and Trained Coffee Tasters. Journal of sensory studies, 25 (2010) 917-939). The test was conducted based on the evaluation criteria by entering absolute evaluations in 9 stages from scores of +4 to −4 based on 0 in a sensory evaluation form by themselves. A comment field was provided in the same sensory evaluation form and the panelists freely remarked their impressions. Smells were evaluated by smelling a sample before the nose, and regarding the other attributes, evaluations were performed by putting the sample in the mouth. Regarding the evaluation results, the data was collected and multiple comparison was then conducted using SPSS statistics 17.0 for Windows (registered trademark) (SPSS Co., Ltd.). Comments of free remarks included responses such as “fragrance that is reminded of rice wine or Amazake (a sweet traditional Japanese drink made from fermented rice)”, “fragrance that gives an alcohol odor” and “good flavor”.

### INDUSTRIAL APPLICABILITY

By preparing self-cloning Aspergillus oryzae obtained by genetic introduction of connecting an amidase gene to the downstream of an enolase promoter gene, the amidase gene can be expressed without induction culture, which thus enables production of a reduced-acrylamide beverage and food.

1. Self-cloning Aspergillus oryzae comprising a sequence which is hybridizable under stringent conditions with a gene that encodes a polypeptide having an amino acid sequence set forth in SEQ ID NO: 1, or a nucleic acid molecule including a base sequence complementary to a gene that encodes the polypeptide, and a gene that encodes a protein having amidase activity introduced therein with the capability of being expressed without induction culture.

2. The self-cloning Aspergillus oryzae of claim 1, wherein the gene is operationally connected to a downstream of an improved enolase promoter.

3. The self-cloning Aspergillus oryzae of claim 1, wherein a specific activity of amidase is at least 27 μmol/min/mg or more.

4. The self-cloning Aspergillus oryzae of claim 1, wherein an expression amount of an amidase gene is at least 2000 times or more as compared to the original strain before self-cloning in a real-time PCR method.

5. A method of reducing acrylamide from an acrylamide-containing matter, comprising a step of subjecting the Aspergillus oryzae of claim 1 to a contact treatment with the acrylamide-containing matter.

6. The method of claim 5, wherein the Aspergillus oryzae is supported on a carrier selected from the group consisting of dried gourd, cellulose, gel beads, porous glass beads, porous ceramics, and unwoven fabric.

7. A method for producing a reduced-acrylamide beverage and food, comprising a step of subjecting the Aspergillus oryzae of claim 1 to a contact treatment with an acrylamide-containing beverage and food.

8. The method of claim 7, wherein the Aspergillus oryzae is supported on a carrier selected from the group consisting of dried gourd, cellulose, gel beads, porous glass beads, porous ceramics, and unwoven fabric.

9. A beverage and food, which has a residual ratio of acrylamide of 50% or less as compared to before treatment due to a contact treatment with the Aspergillus oryzae of claim 1.

10. A beverage and food, comprising increased amounts of 1-propanol, ethyl acetate, 2-methyl-1-butanol, isobutyl alcohol, isoamyl alcohol, ethanol and 2-pentanone respectively twice or more as compared to before treatment due to a contact treatment with self-cloning Aspergillus oryzae.

11. A coffee beverage comprising an acrylamide content of 4 ppb or less.


SEQUENCE LISTING (21 sequences)

SEQ ID NO: 1 - protein, 570 aa, Aspergillus oryzae
MVNVLWKHQGDCSTIVYSCIYHFVTMPSASWEDLAADKRARLEKSIPDEWKFKSVPIEGSVIDLPEKSGILSPSEIKITNSSATELVAQLANGTLKSVDVTLAFCKRAALAHQLVNCAHDFFPELALAQARELDRYFETHKKPVGPLHGLPISLKDQLRVKGTETCMAYISWLGKRDTSDSILTALLRKAGAVFLVKTSVPQTLMVCETVNNIIGRTSNPRNLNLSCGGSSGGEGAMIAMRGGAIGIGTDIGGSIRVPAAFNSLYGIRPSHGRLPYGGMTNSMEGQETIHSVVGPIAHSAQDVRLFLQSVLKEEPWKYDSKVIPLPWREAEENAAQAKIAEKSLNFAFYDFDGVVRPHPPITRGVEIVRSTLEKDGHTVAPWTPYKHAFAVDLANKIYAADGSTDVYKHINASGEPAIPNIKDLMNPNLPKADLNEVWDAQLQKWRYQCEYLDKWREWEERTGKELDAIIAPVAATAAVRHNQFRYYGYATVFNVLDYTSVVVPVTYADKAVDHRLADYQPVSDMDKAVYAEYDPEVYHGAPVAVQIIGRRLSEERTLAIAEYVGKLLGH

SEQ ID NO: 2 - DNA, 2017 bp, Aspergillus oryzae
atggtcaatgttctgtggaaacaccagggtgattgctctaccatcgtctacagctgtatttatcactttgtcactatgccatctgccagctgggaagatctcgctgccgacaagagggcacgtttggagaagtccatccccgacgaatggaaattcaagtcagtcccaatagaaggctcggtcatcgatctacctgagaagtctgggattctgtcgccttctgaaataaagattacaaactcgtctgccacagaacttgtcgctcaattagccaatggcacgttgaagtccgtggatgtgacactcgcattctgtaaaagagctgcactggctcatcaacttgtgggtataaccttcgcctcgatcggagatacatgaaactaatgagaataggttaattgcgcacatgacttcttcccagagctagcactagcccaggccagggaacttgatcggtatttcgagacgcacaagaaacccgtgggaccattgcatggattaccgatttctttgaaagaccaattacgagtcaaggtaagacgagcttcctacactactgtgtgcatctcttctaacatagaactagggaactgaaacatgcatggcctatatctcttggctgggtaagcgcgacaccagcgattcgatattgactgccctcttgagaaaagcgggcgcagtattccttgttaagacgagtgtaccacaaacattgatggtatgtgagaccgtcaataatattatcggtcggacatcgaacccaaggaatctcaacctttcttgcggtggtagttcgggaggcgaaggtgccatgattgcaatgcgtggaggcgccatcggtataggaactgatatcggtaggtatccatacttggttcatcagttattctggcgactaatgatatccaggtggatctattcgtgtcccagccgcattcaactccttgtatgggattcgtccaagtcacggtcgtctgccttacggtggtatgacgaacagcatggaaggtcaggaaacgatacacagcgtcgttggaccaattgcgcattctgctcaaggtagggatctgggatatttcttcgcgtcgagatactgatgctttctagatgtcagactcttccttcagtctgtccttaaggaggaaccttggaagtatgattcgaaagtcataccgcttccttggagggaggccgaggagaacgccgcccaagcaaaaattgctgagaagagtctaaatttcgcattttacgattttgatggcgttgtaagtattagtcgctcctcctccttcgcaatcatgcctgacagttggataaggtacgtcctcaccctccgattactcgtggcgttgagatcgtccggtctacgctcgagaaggacggacataccgtggcaccctggacaccctacaagcatgcatttgccgtagatttagccaacaaaatctacgctgcagatggaagcacggtaagtagcccccctaagaaaattagtatacgtgctaacataatgtaggatgtttacaagcacatcaacgcctcaggagaacccgctattccgaacatcaaggacctcatgaatcccaacctacccaaggcagatttgaatgaggtatgggacgcgcagctgcaaaaatggcgttatcagtgtgaataccttgacaagtggcgcgaatgggaggaacggacgggcaaggagcttgacgctatcatcgccccggtggcggcgacagctgcagtccgccacaaccaattccggtactatgggtatgctactgtctttaacgtgttagattacaccagtgttgttgtcccggttacctatgcagacaaggcggtggatcacagattggcggattatcagccggttagtgatatggataaggcggtttatgcggagtatgatcccgaggtttatcatggcgcacccgttgccgtgcagattatcggcagacgtcttagtgaggagcggaccctggctattgcggagtatgttgggaagttgttaggtcactag

SEQ ID NO: 3 - DNA, 21 bp, Artificial
  Description of Artificial Sequence artificially synthesized prim er sequence
tgtcgctcaattagccaatgg

SEQ ID NO: 4 - DNA, 21 bp, Artificial
  Description of Artificial Sequence artificially synthesized prim er sequence
tgatgagccagtgcagctctt

SEQ ID NO: 5 - DNA, 40 bp, Artificial
  Description of Artificial Sequence artificially synthesized prim er sequence
aaaacacaaagtgaaaggcgccaacgacgactgtctcatt

SEQ ID NO: 6 - DNA, 20 bp, Artificial
  Description of Artificial Sequence artificially synthesized prim er sequence
catggaggggtcatggctac

SEQ ID NO: 7 - DNA, 25 bp, Artificial
  Description of Artificial Sequence artificially synthesized prim er sequence
aaaactcgagaaatactaccttttc

SEQ ID NO: 8 - DNA, 37 bp, Artificial
  Description of Artificial Sequence artificially synthesized prim er sequence
aaaagtcgacaaaagatatccctacgctgattggtcc

SEQ ID NO: 9 - DNA, 20 bp, Artificial
  Description of Artificial Sequence artificially synthesized prim er sequence
gcaaagagagaggaggacga

SEQ ID NO: 10 - DNA, 24 bp, Artificial
  Description of Artificial Sequence artificially synthesized prim er sequence
aaaagtcgactaacttggaggacg

SEQ ID NO: 11 - DNA, 20 bp, Artificial
  Description of Artificial Sequence artificially synthesized prim er sequence
tcaagcgtcgtgtcgggcat

SEQ ID NO: 12 - DNA, 27 bp, Artificial
  Description of Artificial Sequence artificially synthesized prim er sequence
aaaagatatccctacgctgattggtcc

SEQ ID NO: 13 - DNA, 20 bp, Artificial
  Description of Artificial Sequence artificially synthesized prim er sequence
tcgagaaatactaccttttc

SEQ ID NO: 14 - DNA, 65 bp, Artificial
  Description of Artificial Sequence artificially synthesized prim er sequence
aaaagtcgactgaccaattccgcagctcgcgagctaccttgccacgtggtgcgaggagtcgataa

SEQ ID NO: 15 - DNA, 20 bp, Artificial
  Description of Artificial Sequence artificially synthesized prim er sequence
tggtgatgtcatacaaggag

SEQ ID NO: 16 - DNA, 20 bp, Artificial
  Description of Artificial Sequence artificially synthesized prim er sequence
atccgatctcgcagatgtct

SEQ ID NO: 17 - DNA, 47 bp, Artificial
  Description of Artificial Sequence artificially synthesized prim er sequence
agcgaggaagcggaagagcatttaaatgcggttgcgcagttcggtac

SEQ ID NO: 18 - DNA, 38 bp, Artificial
  Description of Artificial Sequence artificially synthesized prim er sequence
acgtggcgccatttaaattgagaactccataaaatgca

SEQ ID NO: 19 - DNA, 39 bp, Artificial
  Description of Artificial Sequence artificially synthesized prim er sequence
atgagacagtcgtcgttggctctgatgctttcgattgtg

SEQ ID NO: 20 - DNA, 25 bp, Artificial
  Description of Artificial Sequence artificially synthesized prim er sequence
tcaaaatggtcaatgttctgtggaa

SEQ ID NO: 21 - DNA, 25 bp, Artificial
  Description of Artificial Sequence artificially synthesized prim er sequence
agcgagtcagaacaaccaataggta